(19) United States
(12) Patent Application Publication
(10) Pub. No.: US 2005/0245456 A1
(43) Pub. Date: Nov. 3, 2005

(54) Combination therapy to prevent and treat diarrhea induced by cancer treatment

(75) Inventors: Diane Healey, Madison, CT (US); Jean Beebe, Salem, CT (US); James J. O'Leary, Mystic, CT (US); W. Gregory Roberts, Noank, CT (US); Anthony Tolcher, San Antonio, TX (US); Mark S. Huberman, Newton, MA (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/119,211

(22) Filed: Apr. 28, 2005

Related U.S. Application Data
(60) Provisional application No. 60/566,213, filed on Apr. 28, 2004.

(51) Int. Cl. A61K 31/425; A61K 38/08; A61K 31/56; A61K 31/138

(52) U.S. Cl. 514/16; 514/171; 514/372; 514/651

(57) ABSTRACT

This invention relates to a method of treating drug side effects in cancer treatment. More particularly, the present invention relates to preventing, reducing, alleviating, or reversing diarrhea caused by the administration of the isothiazole 3-(4-Bromo-2,6-difluoro-benzyloxy)-5-[3-(4-pyrrolidin-1-yl-butyl)-ureido]-isothiazole-4-carboxylic acid amide in the treatment of cancer comprising the step of administering to a patient in need of such treatment, prior to, simultaneously, or sequentially, a therapeutically effective amount of 3-(4-Bromo-2,6-difluoro-benzyloxy)-5-[3-(4-pyrrolidin-1-yl-butyl)-ureido]-isothiazole-4-carboxylic acid amide or a pharmaceutically acceptable salt thereof, and a therapeutically effective amount of an anti-diarrheal agent, a bulking agent, or an anti-estrogen. The combinations of the present invention may optionally include an anti-hypertensive agent.

### BACKGROUND OF THE INVENTION

This invention relates to a combination therapy to treat, prevent, reduce, or decrease diarrhea induced by cancer treatment comprising administering to a patient prior to, simultaneously or sequentially with a therapeutically effective amount of the isothiazole derivative 3-(4-Bromo-2,6-difluoro-benzyloxy)-5-[3-(4-pyrrolidin-1-yl-butyl)-ureido]-isothiazole-4-carboxylic acid amide and a therapeutically effective amount of an anti-diarrheal agent. The methods of the present invention may optionally include an anti-hypertensive agent.

Cancer continues to be one of the leading causes of death in the United States and other developed countries. A side effect of cancer therapy is diarrhea, which often develops during clinical treatment with chemotherapeutic agents. Diarrhea is characterized by the frequent defecation of liquid or liquid-like stools. This adverse effect is most commonly associated with chemotherapeutic agents such as 5-fluorouracil, cisplatin or irinotecan hydrochloride. In particular, late diarrhea due to the administration of chemotherapeutic agents can be prolonged, may lead to dehydration and electrolyte imbalance and can be, in some cases, sufficiently serious that chemotherapeutic agent administration must be modified, interrupted or discontinued.

Diarrhea is a problematic symptom for patients, and because it may provoke reductions in chemotherapeutic agent doses or the frequency of administration, diarrhea may compromise the therapeutic efficacy of chemotherapeutic agents. Late-onset diarrhea, a severe delayed chronic grade 3-4 diarrhea, is a major dose-limiting toxicity in cancer patients. The loss of fluids and electrolytes associated with late diarrhea can result in life-threatening dehydration, renal insufficiency, and electrolyte imbalances. The life-threatening aspects of persistent or severe diarrhea can require aggressive treatment and may lead to hospitalization. Persistent and severe diarrhea can also have a negative effect on the patient's quality of life, interferes with roles and responsibilities, affects interpersonal relationships and promotes feelings of social isolation.

While there is a substantial need for development of an agent for treating cancer therapy induced diarrhea, particularly late diarrhea caused by chemotherapeutic agents, no definite and efficacious method has been identified. The present invention provides a method for treating diarrhea observed after the administration of the isothiazole derivative 3-(4-Bromo-2,6-difluoro-benzyloxy)-5-[3-(4-pyrrolidin-1-yl-butyl)-ureido]-isothiazole-4-carboxylic acid amide. In preventing diarrheal symptoms in patients receiving 3-(4-Bromo-2,6-difluoro-benzyloxy)-5-[3-(4-pyrrolidin-1-yl-butyl)-ureido]-isothiazole-4-carboxylic acid amide, the anti-diarrheal agents of the present invention have the potential to reduce the incidence; severity, and/or duration of diarrhea; improve patient quality of life; avoid hospitalization; and/or prevent dose reduction, interruption, or discontinuation of cancer therapy.

It is therefore the object of the present invention to provide an anti-diarrheal agent for preventing or decreasing diarrhea, in particular late diarrhea, induced by the administration of the isothiazole derivative 3-(4-Bromo-2,6-difluoro-benzyloxy)-5-[3-(4-pyrrolidin-1-y-butyl)-ureido]-isothiazole-4-carboxylic acid amide. The compound 3-(4-Bromo-2,6-difluoro-benzyloxy)-5-[3-(4-pyrrolidin-1-yl-butyl)-ureido]-isothiazole-4-carboxylic acid amide described in U.S. Pat. No. 6,235,764 is hereby incorporated by reference in its entirety.

### SUMMARY OF THE INVENTION

The present invention relates to a method of treating a cancer treatment side effect. More particularly, this invention relates to a combination therapy to treat the drug side effect in cancer treatment of diarrhea comprising administering to a patient prior to, simultaneously or sequentially with a therapeutically effective amount of the isothiazole derivative 3-(4-Bromo-2,6-difluoro-benzyloxy)-5-[3-(4-pyrrolidin-1-yl-butyl)-ureido]-isothiazole-4-carboxylic acid amide or a pharmaceutically acceptable salt, and a therapeutically effective amount of an anti-diarrheal agent selected from the group consisting of Octreotide, Loperamide, bulking agents, Tamoxifen, or an anti-estrogen selected from the group consisting of droloxifene, TAT-59, and raloxifene. The methods of the present invention may optionally include an anti-hypertensive agent.

In one embodiment of the present invention a therapeutically effective amount of 3-(4-Bromo-2,6-difluoro-benzyloxy)-5-[3-(4-pyrrolidin-1-yl-butyl)-ureido]-isothiazole-4-carboxylic acid amide, an analog, a pharmaceutically acceptable salt or a metabolite thereof is administered to a patient being treated prior to, simultaneously, or sequentially with a therapeutically effective amount of an anti-diarrheal agent selected from the list consisting of Octreotide, Loperamide, Tamoxifen, a bulking agent, or an anti-estrogen selected from the group consisting of droloxifene, TAT-59, and raloxifene.

In a preferred embodiment of the invention, the diarrhea is late diarrhea.

In a preferred embodiment, the 3-(4-Bromo-2,6-difluoro-benzyloxy)-5-[3-(4-pyrrolidin-1-yl-butyl)-ureido]-isothiazole-4-carboxylic acid amide is the hydrochloride salt form.

In a preferred embodiment, the 3-(4-Bromo-2,6-difluoro-benzyloxy)-5-[3-(4-pyrrolidin-1-yl-butyl)-ureido]-isothiazole-4-carboxylic acid amide- is the acetate salt form.

In a preferred embodiment, the 3-(4-Bromo-2,6-difluoro-benzyloxy)-5-[3-(4-pyrrolidin-1-yl-butyl)-ureido]-isothiazole-4-carboxylic acid amide is formulated in a long-acting release dosage form.

In a preferred embodiment, the Octreotide is Octreotide Acetate.

In a preferred embodiment, Octreotide Acetate is formulated in a long-acting release formulation.

In a preferred embodiment, the anti-diarrheal agents are selected from the group consisting of Loperamide, bulking agents, and Tamoxifen.

In a preferred embodiment, the anti-diarrheal agents are selected from the group consisting of droloxifene, TAT-59, and raloxifene and administered prior to administration of the 3-(4-Bromo-2,6-difluoro-benzyloxy)-5-[3-(4-pyrrolidin-1-yl-butyl)-ureido]-isothiazole-4-carboxylic acid amide.

In a preferred embodiment, the anti-estrogen is administered about 48 hours prior to 3-(4-Bromo-2,6-difluoro-benzyloxy)-5-[3-(4-pyrrolidin-1-yl-butyl)-ureido]-isothiazole-4-carboxylic acid amide administration.

In a preferred embodiment, the anti-estrogen is administered at a time effective to impede intestinal epithelial cell replication in the G₀/G₁ phase of the cell cycle.

In a preferred embodiment, the anti-estrogen is administered to a prior to administration of the 3-(4-Bromo-2,6-difluoro-benzyloxy)-5-[3-(4-pyrrolidin-1-yl-butyl)-ureido]-isothiazole-4-carboxylic acid amide.

In a preferred embodiment, the anti-estrogen is administered to said patient about at least 48 hours prior to administration of the 3-(4-Bromo-2,6-difluoro-benzyloxy)-5-[3-(4-pyrrolidin-1-yl-butyl)-ureido]-isothiazole-4-carboxylic acid amide.

In a preferred embodiment, the amount of anti-estrogen administered to a patient is in the range of about 0.4-0.8 mg/kg/day.

In a preferred embodiment, the anti-diarrheal agent, Tamoxifen, is administered prior to administration of the 3-(4-Bromo-2,6-difluoro-benzyloxy)-5-[3-(4-pyrrolidin-1-yl-butyl)-ureido]-isothiazole-4-carboxylic acid amide.

In a preferred embodiment, Tamoxifen is administered about 48 hours prior to the administration of 3-(4-Bromo-2,6-difluoro-benzyloxy)-5-[3-(4-pyrrolidin-1-yl-butyl)-ureido]-isothiazole-4-carboxylic acid amide.

In a preferred embodiment, the administration of Tamoxifen is at a time effective to impede intestinal epithelial cell replication in the G₀/G₁ phase of the cell cycle.

In a preferred embodiment, the Tamoxifen is administered to a patient about at least 48 hours prior to administration of the 3-(4-Bromo-2,6-difluoro-benzyloxy)-5-[3-(4-pyrrolidin-1-yl-butyl)-ureido]-isothiazole-4-carboxylic acid amide.

In a preferred embodiment, the Tamoxifen is administered to a patient in the range of about 0.4-0.8 mg/kg/day.

In one embodiment of the method of the present invention the cancer is selected from the group consisting of brain, squamous cell, bladder, gastric, pancreatic, breast, head, neck, esophageal, prostate, colorectal, lung, renal, kidney, ovarian, gynecological and thyroid cancer.

In one preferred embodiment the cancer is selected from the group consisting of prostate, breast, lung, colon or ovarian cancer.

In a more preferred embodiment the cancer is selected from the group consisting of prostate, breast, and lung cancer.

In one preferred embodiment the breast cancer is metastatic breast cancer.

In another preferred embodiment the lung cancer is non-small cell lung cancer (NSCL).

In one embodiment of the present invention the anti-hypertensive agent is selected from the group consisting of calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers (α-blockers).

In a preferred embodiment of the present invention the anti-hypertensive agent is an angiotensin converting enzyme inhibitor (ACE inhibitor).

In one embodiment the ACE inhibitor is accupril (quinapril) or accuretic (quinapril and hydrochlorothiazide).

In another preferred embodiment of the present invention the anti-hypertensive agent is an alpha-adrenergic receptor blocker (α-blocker).

In one embodiment of the present invention the alpha-adrenergic receptor blocker (α-blocker) is selected from the group consisting of cardura (doxazosin) or cardura XL (doxazosin GITS).

In another preferred embodiment of the present invention the anti-hypertensive agent is a calcium channel blocker.

In one embodiment the calcium channel blocker is selected from the group consisting of Norvasc (amlodipine), procardia (nifedipine), and procardia XL (nifedipine GITS).

The present invention also relates to a pharmaceutical composition for the treatment of cancer in a patient which comprises a therapeutically effective amount of Octreotide, Loperamide, Tamoxifen, a bulking agent, an anti-estrogen selected from the group consisting of droloxifene, TAT-59, or raloxifene and a therapeutically effective amount of 3-(4-Bromo-2,6-difluoro-benzyloxy)-5-[3-(4-pyrrolidin-1-yl-butyl)-ureido]-isothiazole-4-carboxylic acid amide, in combination with one or more pharmaceutically acceptable carriers or vehicles.

The present invention also relates to a pharmaceutical composition for the treatment of cancer in a patient which comprises a therapeutically effective amount of Octreotide, Loperamide, Tamoxifen, a bulking agent, an anti-estrogen selected from the group consisting of droloxifene, TAT-59, or raloxifene and a therapeutically effective amount of 3-(4-Bromo-2,6-difluoro-benzyloxy)-5-[3-(4-pyrrolidin-1-yl-butyl)-ureido]-isothiazole-4-carboxylic acid amide and a therapeutically effective amount of an anti-hypertensive agent, in combination with one or more pharmaceutically acceptable carriers or vehicles.

In one preferred embodiment the pharmaceutical composition is for the treatment of cancer selected from brain, squamous cell, bladder, gastric, pancreatic, breast, head, neck, esophageal, prostate, colorectal, lung, renal, kidney, ovarian, gynecological and thyroid cancer. In a more preferred embodiment the pharmaceutical composition is for the treatment of prostate, breast, lung, colon and ovarian cancer. In an even more preferred embodiment the pharmaceutical composition is for the treatment of prostate, breast, and lung cancer. In a most preferred embodiment the pharmaceutical composition is for the treatment of metastatic breast cancer or NSCL.

The present invention also relates to a kit comprising in a first compartment a therapeutically effective amount of 3-(4-Bromo-2,6-difluoro-benzyloxy)-5-[3-(4-pyrrolidin-1-yl-butyl)-ureido]-isothiazole-4-carboxylic acid amide and in a second compartment a therapeutically effective amount of Octreotide, Loperamide, Tamoxifen, a bulking agent, droloxifene, TAT-59, or raloxifene.

The present invention also relates to a kit comprising a first compartment containing a therapeutically effective amount of 3-(4-Bromo-2,6-difluoro-benzyloxy)-5-[3-(4-pyrrolidin-1-yl-butyl)-ureido]-isothiazole-4-carboxylic acid amide a second compartment containing a therapeutically effective amount of Octreotide, Loperamide, Tamoxifen, a bulking agent, droloxifene, TAT-59, or raloxifene, and a third compartment containing an anti-hypertensive agent.

In one embodiment of the kit of the present invention the compound in the second compartment is Octreotide.

In another embodiment of the kit of the present invention the second compartment is Loperamide.

In another preferred embodiment of the kit of the present invention the compound in the second compartment is Tamoxifen.

In one preferred embodiment of the kit of the present invention the compound in the second compartment is an anti-estrogen selected from the group consisting of droloxifene, TAT-59, or raloxifene.

In another preferred embodiment of the kit of the present invention the second compartment is droloxifene.

In another preferred embodiment of the kit of the present invention the second compartment is TAT-59.

In another preferred embodiment of the kit of the present invention the second compartment is raloxifene.

### DETAILED DESCRIPTION OF THE INVENTION

The present invention relates to a method of treating diarrhea in a patient which comprises administering to said patient in need of such treatment, prior to, simultaneously, or sequentially with a therapeutically effective amount of the isothiazole derivative 3-(4-Bromo-2,6-difluoro-benzyloxy)-5-[3-(4-pyrrolidin-1-yl-butyl)-ureido]-isothiazole-4-carboxylic acid amide and a therapeutically effective amount of Octreotide, Loperamide, Tamoxifen, a bulking agent, or an anti-estrogen selected from the group consisting of droloxifene, TAT-59, and raloxifene. The methods of the present invention may optionally include an anti-hypertensive agent.

The isothiazole compound of the present invention and its pharmaceutically acceptable salts and solvates may be prepared as described in U.S. Pat. No. 6,235,764, the contents of which are incorporated by reference.

One embodiment of the combination therapy of the present invention includes Octreotide. Octreotide is the acetate salt of a cyclic octapeptide. It is a long-acting octapeptide with pharmacologic properties mimicking those of the natural hormone somatostatin. Octreotide has been used in co-therapy for ameliorating delayed diarrhea (Cascinu S. Management of diarrhea induced by tumors or cancer therapy. Curr. Opin. Oncol. 1995;7:325-329). In 1990, Kennedy et al. reported encouraging results of treatment with Octreotide in 11 patients with colorectal cancer suffering from diarrhea after chemotherapy with 5-fluorouracil (Proc. Am. Soc. Clin. Oncol., 1990 9:324). These data were confirmed in other pilot trials (Cascinu S. et al., Eur. J. Cancer, 1992 28-482-483; and Petrelli N. et al., Cancer 1993, 75-1543-1546) and in a randomized trial comparing Octreotide with Loperamide in the therapy of 5-fluorouracil-induced diarrhea, in which Octreotide was shown to be more effective than Loperamide and probably more cost-effective (Cascinu S. et al., J. Clin. Oncol., 1993, 11:148-151). Octreotide has been found to be active in preventing diarrhea associated with the administration of cisplatin in patients who experienced this side-effect during a previous course of chemotherapy (Cascinu S., Fedeli A., Luzi Fedeli S. and Catalano G., Oncology, 1994;51:70-73).

Alternatively, the combination therapy of the present invention can include Loperamide. In humans, intensive, immediate application of Loperamide (an agent that slows intestinal motility and affects water and electrolyte movement through the bowel) has been used to reduce or control diarrhea once diarrhea has started (Rougier P, Bugat R. CPT-11 in the treatment of colorectal cancer: Clinical efficacy and safety profile. Semin Oncol 1996;23(Suppl 3): 34-41.

Another embodiment of the combination therapy of the present invention includes Tamoxifen. Tamoxifen (Nolvadex®) interferes with the activity of the hormone, estrogen. Estrogen promotes the growth of breast cancer cells. Tamoxifen works against the effects of estrogen on these cells. It is often called an “anti-estrogen. Tamoxifen, a marketed antiestrogen chemotherapeutic agent, is known to induce a block in the G0/G1 phase of the cell cycle. Unlike most chemotherapeutics, which act in the S, G2 and M phases of the cell cycle, Tamoxifen clearly blocks cell cycle progression in G0/G1 (Otto A M, Paddenberg R, Schubert S, Mannherz H G. Cell cycle arrest, micronucleus formation, and cell death in growth inhibition of MCF-7 breast cancer cells by tamoxifen and cisplatin. J Canc Res & Clin Oncol 1996;122:603-612). This is thought to be through repression of G1-specific protein kinase activity. PCT application WO/96/01127 (published Jan. 18, 1996) discloses a wide variety of agents to be co-administered with Irinotecan, including Tamoxifen. However, it does not disclose prior administration of tamoxifen. The use of Tamoxifen as a therapy to reduce Irinotecan Hydrochloride-induced diarrhea is claimed in U.S. Pat. 6,087,377. One embodiment of the combination therapy of the present invention includes bulking agents. Bulking agents, such as Metamucil and Citrucel contain high amounts of fiber and help to slow down diarrhea.

Another embodiment of the combination therapy of the present invention includes an anti-estrogen selected from the group consisting of droloxifene, TAT-59, and raloxifene (Evista). Raloxifene is a selective estrogen receptor modulator that produces both estrogen-agonistic effects on bone and lipid metabolism and estrogen-antagonistic effects on uterine endometrium and breast tissue. Because of its tissue selectivity, raloxifene may have fewer side effects than are typically observed with estrogen therapy. Raloxifene (Evista) has the ability to bind to and activate the estrogen receptor while exhibiting tissue-specific effects distinct from estradiol. As a result, raloxifene is the first of a benzothiophene series of anti-estrogens to be labeled a SERM.

The present invention also relates to a method of treating diarrhea in a patient which comprises administering to said patient in need of such treatment, prior to, simultaneously, or sequentially, a therapeutically effective amount of 3-(4-Bromo-2,6-difluoro-benzyloxy)-5-[3-(4-pyrrolidin-1-yl-butyl)-ureido]-isothiazole-4-carboxylic acid amide and a therapeutically effective amount of Octreotide, Loperamide, Tamoxifen, a bulking agent, an anti-estrogen selected from the group consisting of droloxifene, TAT-59, and a therapeutically effective amount of an anti-hypertensive agent. When combinations of the present invention are administered sequentially each agent may be administered, first, second or third. In one preferred embodiment, the agents of the combination are administered as (i), the 3-(4-Bromo-2,6-difluoro-benzyloxy)-5-[3-(4-pyrrolidin-1-yl-butyl)-ureido]-isothiazole-4-carboxylic acid amide followed by a therapeutically effective amount of Octreotide, Loperamide, Tamoxifen, a bulking agent, an anti-estrogen selected from the group consisting of droloxifene, TAT-59, and raloxifene and followed by the anti-hypertensive agent.

Combinations of the invention may be administered sequentially or may be administered simultaneously.

The term “anti-hypertensive” means any agent, which lowers blood pressure. There are many different categories of anti-hypertensive agents including calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers (α-blockers). Any anti-hypertensive agent may be used in accordance with this invention and examples from each class are given hereinafter.

Calcium channel blockers, which are within the scope of this invention include, but are not limited to: Norvasc (amlodipine) (U.S. Pat. No. 4,572,909); procardia (nifedipine), procardia XL (nifedipine GITS); bepridil (U.S. Pat. No. 3,962,238 or U.S. Reissue No. 30,577); clentiazem (U.S. Pat. No. 4,567,175); diltiazem (U.S. Pat. No. 3,562,257); fendiline (U.S. Pat. No. 3,262,977); gallopamil (U.S. Pat. No. 3,261,859); mibefradil (U.S. Pat. No. 4,808,605); prenylamine (U.S. Pat. No. 3,152,173); semotiadil (U.S. Pat. No. 4,786,635); terodiline (U.S. Pat. No. 3,371,014); verapamil (U.S. Pat. No. 3,261,859); aranidipine (U.S. Pat. No. 4,446,325); bamidipine (U.S. Pat. No. 4,220,649); benidipine (European Patent Application Publication No. 106,275); cilnidipine (U.S. Pat. No. 4,672,068); efonidipine (U.S. Pat. No. 4,885,284); elgodipine (U.S. Pat. No. 4,952,592); felodipine (U.S. Pat. No. 4,264,611); isradipine (U.S. Pat. No. 4,466,972); lacidipine (U.S. Pat. No. 4,801,599); lercanidipine (U.S. Pat. No. 4,705,797); manidipine (U.S. Pat. No. 4,892,875); nicardipine (U.S. Pat. No. 3,985,758); nifedipine (U.S. Pat. No. 3,485,847); nilvadipine (U.S. Pat. No. 4,338,322); nimodipine (U.S. Pat. No. 3,799,934); nisoldipine (U.S. Pat. No. 4,154,839); nitrendipine (U.S. Pat. No. 3,799,934); cinnarizine (U.S. Pat. No. 2,882,27 1); flunarizine (U.S. Pat. No. 3,773,939); lidoflazine (U.S. Pat. No. 3,267,104); lomerizine (U.S. Pat. No. 4,663,325); bencyclane (Hungarian Patent No. 151,865); etafenone (German Patent No. 1,265,758); and perhexiline (British Patent No. 1,025,578). The disclosures of all such patents and patent applications are incorporated herein by reference.

Angiotensin Converting Enzyme Inhibitors (ACE-Inhibitors) which are within the scope of this invention include, but are not limited to: accupril (quinapril); accuretic (quinapril and hydrochlorothiazide); alacepril (U.S. Pat. No. 4,248,883); benazepril (U.S. Pat. No. 4,410,520); captopril (U.S. Pat. Nos. 4,046,889 and 4,105,776); ceronapril (U.S. Pat. No. 4,452,790); delapril (U.S. Pat. No. 4,385,051); enalapril (U.S. Pat. No. 4,374,829); fosinopril (U.S. Pat. No. 4,37,201); imidapril (U.S. Pat. No. 4,508,727); lisinopril (U.S. Pat. No. 4,555,502); moveltipril (Belgium Patent No. 893,553); perindopril (U.S. Pat. No. 4,508,729); quinapril (U.S. Pat. No. 4,344,949); ramipril (U.S. Pat. No. 4,587,258); spirapril (U.S. Pat. No. 4,470,972); temocapril (U.S. Pat. No. 4,699,905); and trandolapril (U.S. Pat. No. 4,933,361). The disclosures of all such patents are incorporated herein by reference.

Angiotensin-II receptor antagonists (A-II antagonists) which are within the scope of this invention include, but are not limited to: candesartan (U.S. Pat. No. 5,196,444); eprosartan (U.S. Pat. No. 5,185,351); irbesartan (U.S. Pat. No. 5,270,317); losartan (U.S. Pat. No. 5,138,069); and valsartan (U.S. Pat. No. 5,399,578). The disclosures of all such U.S. patents are incorporated herein by reference.

β-Blockers which are within the scope of this invention include, but are not limited to: acebutolol (U.S. Pat. No. 3,857,952); alprenolol (Netherlands Patent Application No. 6,605,692); amosulalol (U.S. Pat. No. 4,217,305); arotinolol (U.S. Pat. No. 3,932,400); atenolol (U.S. Pat. Nos. 3,663,607 and 3,836,671); befunolol (U.S. Pat. No. 3,853,923); betaxolol (U.S. Pat. No. 4,252,984); bevantolol (U.S. Pat. No. 3,857,891); bisoprolol (U.S. Pat. No. 4,258,062); bopindolol (U.S. Pat. No. 4,340,541); bucumolol (U.S. Pat. No. 3,663,570); bufetolol (U.S. Pat. No. 3,723,476); bufuralol (U.S. Pat. No. 3,929,836); bunitrolol (U.S. Pat. No. 3,541,130); bupranolol (U.S. Pat. No. 3,309,406); butidrine hydrochloride (French Patent No. 1,390,056); butofilolol (U.S. Pat. No. 4,302,601); carazolol (German Patent No. 2,240,599); carteolol (U.S. Pat. No. 3,910,924); carvedilol (U.S. Pat. No. 4,503,067); celiprolol (U.S. Pat. No. 4,034,009); cetamolol (U.S. Pat. No. 4,059,622); cloranolol (German Patent No. 2, 213,044); dilevalol (Clifton et al., Journal of Medicinal Chemistry, 1982, 25, 670); epanolol (U.S. Pat. No. 4,167,58 1); indenolol (U.S. Pat. No. 4,045,482); labetalol (U.S. Pat. No. 4,012,444); levobunolol (U.S. Pat. No. 4,463,176); mepindolol (Seeman et al, Helv. Chim. Acta, 1971, 54, 2411); metipranolol (Czechoslovakian Patent Application No. 128,471); metoprolol (U.S. Pat. No. 3,873,600); moprolol (U.S. Pat. No. 3,501,769); nadolol (U.S. Pat. No. 3,935,267); nadoxolol (U.S. Pat. No. 3,819,702); nebivalol (U.S. Pat. No. 4,654,362); nipradilol (U.S. Pat. No. 4,3 94,382); oxprenolol (British Patent No. 1,077,603); penbutolol (U.S. Pat. No. 3,551,493); pindolol (Swiss Patents Nos. 469,002 and 472,404); practolol (U.S. Pat. No. 3,408,387); pronethalol (British Patent No. 909,357); propranolol (U.S. Pat. Nos. 3,337,628 and 3,520,919); sotalol (Uloth et al., Journal of Medicinal Chemistry, 1966, 9, 88); sulfinalol (German Patent No. 2,728,641); talinolol (U.S. Pat. Nos. 3,935,259 and 4,038,313); tertatolol (U.S. Pat. No. 3,960,891); tilisolol (U.S. Pat. No. 4,129,565); timolol (U.S. Pat. No. 3,655,663); toliprolol (U.S. Pat. No. 3,432,545); and xibenolol (U.S. Pat. No. 4,018,824. The disclosures of all such patents, patent applications and references are incorporated herein by reference.

α-Blockers which are within the scope of this invention include, but are not limited to: cardura (doxazosin); cardura XL (doxazosin GITS); amosulalol (U.S. Pat. No. 4,217,305); arotinolol; dapiprazole (U.S. Pat. No. 4,252,721); doxazosin (U.S. Pat. No. 4,188,390); fenspiride (U.S. Pat. No. 3,399,192); indoramin (U.S. Pat. No. 3,527,761); labetolol, naftopidil (U.S. Pat. No. 3,997,666); nicergoline (U.S. Pat. No. 3,228,943); prazosin (U.S. Pat. No. 3,511,836); tainsulosin (U.S. Pat. No. 4,703,063); tolazoline (U.S. Pat. No. 2,161,93 8); trimazosin (U.S. Pat. No. 3,669,968); and yohimbine, which may be isolated from natural sources according to methods well known to those skilled in the art. The disclosures of all such U.S. patents are incorporated herein by reference.

Preferred anti-hypertensive agents of the invention include calcium channel blockers, alpha-adrenergic blockers, and ACE inhibitors.

The anti-hypertensives described herein are generally commercially available, or they may be made by standard techniques including those described in the references cited herein.

The term “treating”, as used herein, refers to reversing, decreasing, alleviating, inhibiting the progress of, or preventing the disorder or condition to which such term applies, or one or more symptoms of such disorder or condition. The term “treatment”, as used herein, refers to the act of treating, as “treating” is defined immediately above.

The phrase “pharmaceutically acceptable salt(s)”, as used herein, unless otherwise indicated, includes salts of acidic or basic groups of 3-(4-Bromo-2,6-difluoro-benzyloxy)-5-[3-(4-pyrrolidin-1-yl-butyl)-ureido]-isothiazole-4-carboxylic acid amide. This compound which is basic in nature is capable of forming a wide variety of salts with various inorganic and organic acids. The acids that may be used to prepare pharmaceutically acceptable acid addition salts of this compound are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

The terms “concurrently” and “simultaneously” are used interchangeably and mean the compounds of the combination therapy of the present invention are administered (1) simultaneously in time, or (2) at different times during the course of a common treatment schedule.

The term “sequentially” as used herein means administration of 3-(4-Bromo-2,6-difluoro-benzyloxy)-5-[3-(4-pyrrolidin-1-yl-butyl)-ureido]-isothiazole-4-carboxylic acid amide or of Octreotide, Loperamide, Tamoxifen, droloxifene, TAT-59, or raloxifene followed by administration of the other component; after administration of one component, the second component can be administered substantially immediately after the first component, or the second component can be administered after an effective time period after the first component; the effective time period is the amount of time given for realization of maximum benefit from the administration of the first component.

Administration of the compounds of the present invention can be effected by any method that enables delivery of the compounds to the site of action. These methods include oral routes, intraduodenal routes, parenteral injection (including intravenous, subcutaneous, intramuscular, intravascular or infusion), intraocular, intraperitoneal, intravesicular, intravaginal, topical, and rectal administration.

The amount of the active compounds administered will depend on the subject being treated, the severity of the diarrhea, or the rate of administration and the judgement of the prescribing physician.

The products of which the combination are composed may be administered simultaneously, separately or spaced out over a period of time so as to obtain the maximum efficacy of the combination; it being possible for each administration to vary in its duration from a rapid administration to a continuous perfusion. As a result, for the purposes of the present invention, the combinations are not exclusively limited to those which are obtained by physical association of the constituents, but also to those which permit a separate administration, which can be simultaneous or spaced out over a period of time. The compositions according to the invention are preferably compositions which can be administered parentally. However, these compositions may be administered orally or intraperitoneally in the case of localized regional therapies.

The compositions for parental administration are generally pharmaceutically acceptable, sterile solutions or suspensions which may optionally be prepared as required at the time of use. For the preparation of non-aqueous solutions or suspensions, natural vegetable oils such as—olive oil, sesame oil or liquid petroleum or injectable organic esters such as ethyl oleate may be used. The sterile aqueous solutions can consist of a solution of the product in water. The aqueous solutions are suitable for intravenous administration provided the pH is appropriately adjusted and the solution is made isotonic, for example with a sufficient amount of sodium chloride or glucose. The sterilization may be carried out by heating or by any other means which does not adversely affect the composition.

The combinations may also take the form of liposomes or the form of an association with carriers as cyclodextrins or polyethylene glycols. The compositions for oral or intraperitoneal administration are preferably aqueous suspensions or solutions.

The combinations of the present invention are formulated alone, however they may also be formulated together if desired. This facilitates the easy of use (i.e., less tablets for a patient to swallow) and patient compliance since one tablet is a desired dosage form.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The example provided below illustrates and exemplifies a method of administration of the combination therapy of the present invention. It is to be understood that the scope of the present invention is not limited in any way by the scope of the following prophetic example.

### EXAMPLE 1

In a Phase 1 trial with 3-(4-Bromo-2,6-difluoro-benzyloxy)-5-[3-(4-pyrrolidin-1-yl-butyl)-ureido]-isothiazole-4-carboxylic acid amide HCl almost all subjects treated with doses greater than 160 mg/day may develop Grade 1 or 2 diarrhea within the first few days. Susceptible subjects are encouraged to adhere to a daily schedule of prophylactic anti-diarrheal agents, such as Loperamide, Octreotide, Loperamide, Tamoxifen, a bulking agent, or an anti-estrogen. To treat the diarrhea in these patients, a therapeutically effective amount of 3-(4-Bromo-2,6-difluoro-benzyloxy)-5-[3-(4-pyrrolidin-1-yl-butyl)-ureido]-isothiazole-4-carboxylic acid amide HCl is administered to a patient in need of such therapy, prior to, simultaneously or sequentially with a therapeutically effective amount of Loperamide, Octreotide, Loperamide, Tamoxifen, a bulking agent, or an anti-estrogen.

The following table shows a recommended treatment for various stages of diarrhea induced by 3-(4-Bromo-2,6-difluoro-benzyloxy)-5-[3-(4-pyrrolidin-1-yl-butyl)-ureido]-isothiazole-4-carboxylic acid amide HCl cancer therapy.

TABLE 1
Intervention for Diarrhea

| Severity of Diarrhea | CTC Grade | Intervention |
|---|---|---|
| <4 stools/day | 1 | Use anti-diarrheal agent at |
| over baseline | | subject's own discretion according |
| | | to their comfort level |
| Increase of 4-6 stools/ | 2 | Recommend prophylactic anti- |
| day or nocturnal stools | | diarrheal agent on a daily basis in |
| | | combination with cancer therapy |

1. A method of treating diarrhea in a patient with cancer which comprises administering to said patient, prior to, simultaneously or sequentially a therapeutically effective amount of 3-(4-Bromo-2,6-difluoro-benzyloxy)-5-[3-(4-pyrrolidin-1-yl-butyl)-ureido]-isothiazole-4-carboxylic acid amide or a pharmaceutically acceptable salt thereof, and a therapeutically effective amount of an anti-diarrheal agent.

2. The method according to claim 1 wherein the anti-diarrheal agent is selected from the group consisting of Octreotide, Loperamide, Tamoxifen, a bulking agent, or an anti-estrogen.

3. The method according to claim 2, wherein the anti-estrogen is selected from the group consisting of droloxifene, TAT-59, and raloxifene.

4. The method of claim 1 wherein said cancer is selected from the group consisting of brain, squamous cell, bladder, gastric, pancreatic, breast, head, neck, esophageal, prostate, colorectal, lung, renal, kidney, ovarian, gynecological and thyroid cancer.

5. The method of claim 1, wherein said anti-diarrheal agents are administered prior to administration of said 3-(4-Bromo-2,6-difluoro-benzyloxy)-5-[3-(4-pyrrolidin-1-yl-butyl)-ureido]-isothiazole-4-carboxylic acid amide.

6. The method of claim 1, wherein said anti-diarrheal agents are administered simultaneously with said 3-(4-Bromo-2,6-difluoro-benzyloxy)-5-[3-(4-pyrrolidin-1-yl-butyl)-ureido]-isothiazole-4-carboxylic acid amide.

7. The method of claim 1, wherein said anti-diarrheal agents are administered after administration of said 3-(4-Bromo-2,6-difluoro-benzyloxy)-5-[3-(4-pyrrolidin-1-yl-butyl)-ureido]-isothiazole-4-carboxylic acid amide.

8. A kit comprising in a first compartment 3-(4-Bromo-2,6-difluoro-benzyloxy)-5-[3-(4-pyrrolidin-1-yl-butyl)-ureido]-isothiazole-4-carboxylic acid amide and in a second compartment a therapeutically effective amount of Octreotide, Loperamide, Tamoxifen, a bulking agent, or an anti-estrogen selected from the group consisting of droloxifene, TAT-59, and raloxifene.

9. A method of treating diarrhea in a patient with cancer which comprises administering to said patient in need of such treatment, prior to, simultaneously or sequentially a therapeutically effective amount of 3-(4-Bromo-2,6-difluoro-benzyloxy)-5-[3-(4-pyrrolidin-1-yl-butyl)-ureido]-isothiazole-4-carboxylic acid amide or a pharmaceutically acceptable salt thereof, and a therapeutically effective amount of Octreotide, Loperamide, Tamoxifen, a bulking agent, or an anti-estrogen selected from the group consisting of droloxifene, TAT-59, and raloxifene and a therapeutically effective amount of an anti-hypertensive agent.

10. The method according to claim 9, wherein the anti-hypertensive agent is selected from the group consisting of calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers (α-blockers).

11. The method according to claim 1, wherein the diarrhea is late diarrhea.

12. The method according to claim 1, wherein the 3-(4-Bromo-2,6-difluoro-benzyloxy)-5-[3-(4-pyrrolidin-1-yl-butyl)-ureido]-isothiazole-4-carboxylic acid amide is the hydrochloride salt form.

13. The method according to claim 1, wherein the 3-(4-Bromo-2,6-difluoro-benzyloxy)-5-[3-(4-pyrrolidin-1-yl-butyl)-ureido]-isothiazole-4-carboxylic acid amide is the acetate salt form.

14. The method according to claim 1 wherein the 3-(4-Bromo-2,6-difluoro-benzyloxy)-5-[3-(4-pyrrolidin-1-yl-butyl)-ureido]-isothiazole-4-carboxylic acid amide is formulated in a long-acting release dosage form.
